(19) United States
(12) Patent Application Publication
(10) Pub. No.: US 2017/0246401 A1
(43) Pub. Date: Aug. 31, 2017

(54) INDEXING DELIVERY DEVICE

(71) Applicant: WestRock Dispensing Systems, Inc., Norcross, GA (US)

(72) Inventor: Joseph F. KEENAN, Superior, CO (US)

(73) Assignee: WestRock Dispensing Systems, Inc., Norcross, GA (US)

(21) Appl. No.: 15/516,393

(22) PCT Filed: Oct. 7, 2015
(86) PCT No.: PCT/US15/54441

§ 371 Date: Apr. 1, 2017

Related U.S. Application Data
(60) Provisional application No. 62/060,870, filed on Oct. 7, 2014.

(51) Int. Cl. A61M 5/315; A61J 7/00; A61M 39/24

(52) U.S. Cl. CPC A61M 5/31593; A61M 5/31551; A61M 39/24; A61J 7/0046; A61M 2005/3126

(57) ABSTRACT

A dispensing device is disclosed which provides for dispensing multiple doses in amounts determined by a movable collar attached to a plunger shaft.

### BACKGROUND OF THE INVENTION

The present application is directed to a device for dispensing multiple, sequential amounts of liquids or fluids including semi-solid fluids, such as medication doses, precise aliquots of adhesives, or other useful fluids where a consistent and repeatable dispensing amount is desired.

Products such as fluid medicines, for example cough syrups and the like, may be packaged in containers holding multiple doses that are to be dispensed by pouring the medicine into a spoon or into a small cup. Such methods are prone to spilling and subject to inexact amounts. A more repeatable, neater dispensing method is desired.

It is known to have dispensing devices capable of delivering several measured doses. For example, United States Patent Application Publication 20130289493 to Baney et al discloses a “Dose dividing delivery device,” but the device is complex. It would be advantageous to have a delivery device that is simple to manufacture and use. The dispensing device disclosed here has a simple construction that facilitates its use as a disposable, pre-filled dispensing device.

### SUMMARY

In one aspect, a fluid dispensing device is disclosed which includes a cylinder, a plunger movable through said cylinder, the plunger having a plurality of radial ledges along the plunger length and a stopper attached to an inner end of the plunger, the stopper closely fitting an inside wall of the cylinder, and a collar fitted around the plunger, the collar having at least one passage for movement therethrough of a radial ledge.

In certain embodiments, the fluid dispensing device may include a plurality of radial ledges equally spaced along the plunger length.

In certain embodiments, the spacing of radial ledges along the plunger length corresponds to a fluid dosage to be delivered.

In certain embodiments, the plurality of radial ledges alternate between different angular positions around the plunger.

In certain embodiments, the angular positions of consecutive radial ledges along the plunger length alternate by 90 degrees around the plunger.

In certain embodiments, the fluid dispensing device further includes an anti-reverse feature to prevent the plunger from moving outward from the cylinder.

Other aspects of the disclosed fluid dispensing device will become apparent from the following description and the accompanying drawings.

### BRIEF DESCRIPTION OF THE DRAWINGS

FIG. 1 shows a side view of a fluid dispensing device;

FIG. 2 shows a side view of the parts of the fluid dispensing device;

FIG. 3 shows a perspective view of the parts of the fluid dispensing device; and

FIG. 4 shows several views of the fluid dispensing device in use.

### DETAILED DESCRIPTION

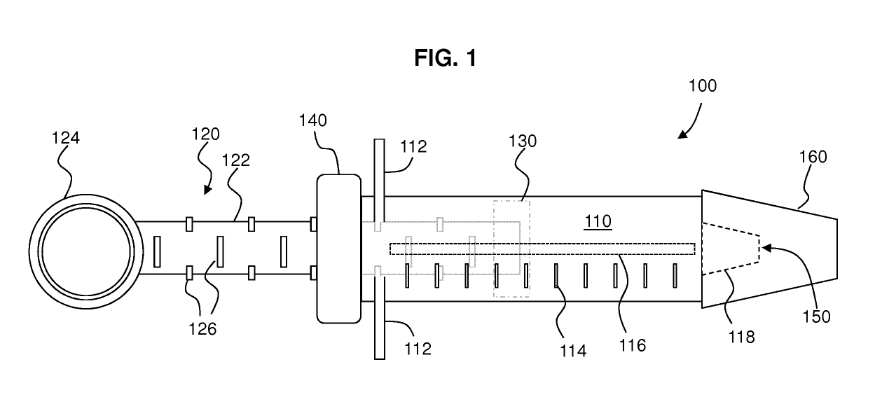

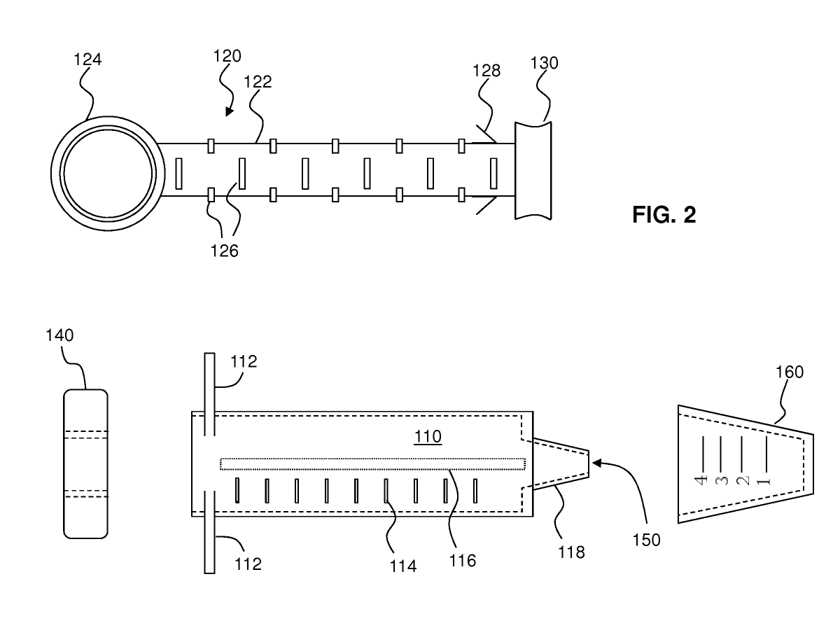

As various embodiments of the fluid dispensing device are described, reference will be made to FIGS. 1-5. Certain parts of the dispensing device are denoted by reference numerals. Where there is more than one of the same feature, generally only one will be denoted by a reference numeral. Where assembly steps are described, these steps are exemplary and are not to be limiting as to the sequence of operations used to arrive at the final package. Also, directions such as up, down, top, bottom, front, back, etc. are used for convenience in describing the device and are not meant to be limiting.

FIG. 1 shows a side view of a dispensing device 100, in an assembled state. The parts include cylinder 110, plunger 120, stopper 130 (hidden within the cylinder), collar 140, outlet valve 150, and cap 160.

Cylinder 110 may by a generally right circular shape, such as a round tube. The cylinder may be made of a material such as a plastic and may be formed by injection molding. The cylinder may have an open end (toward the left in FIG. 1) and distal end (toward the right in FIG. 1). Near the open end there may be provided a flange 112 to facilitate holding the cylinder. Along at least part of the length of cylinder 110 may be provided gradations 114 such as printed indicia, molded indicia, or transparent, translucent, or opaque areas.

Cylinder 110 may be transparent, translucent, or opaque. If the cylinder is not transparent, at least a portion 116 may be provided which is transparent or translucent in order for a user to see the contents of the cylinder. At the distal end of the cylinder there may be provided an outlet such as nozzle 118. A one way valve or drip-prevention feature such as a slit valve or duckbill valve 150 or split or rolling septum may be provided within or on the outlet nozzle 118. Alternately, a spring-loaded valve may be used. Such an outlet valve may prevent dripping of the dispenser, or contamination of its contents, during non-use times. An outlet valve, for example a spring-loaded valve, may prevent a child from sucking the contents from the cylinder, and may provide an additional microbial barrier for the contents. A removable cap 160 may be provided to attach onto or fit over the distal end of cylinder 110 and/or nozzle 118. Cap 160 may be used as an auxiliary dispensing device, for example with the dose dispensed into the cap so that it may be poured from the cap into a patient's mouth. The cap may have graduations provided thereon, such as by molding or printing. The cap may be attached to cylinder 110 or nozzle 118 by a child-resistant feature, for example using any of the known child-resistant attachments by which a cap may be attached to a medicine bottle.

Plunger 120 may be sized to fit in cylinder 110. The plunger shaft 122 may be about the same length as the cylinder 110, and may have a diameter, including radial ledges 126, of not more than the inner diameter of cylinder 110.

The outer end of plunger 120 may be a thumb-engaging feature such as ring 124, or a button, disk, or other shape on which the user may push to move the plunger 120 into cylinder 110.

Radial ledges 126 may be provide along the plunger shaft 122. Preferably ledges 126 may be equally spaced along the length of the shaft. The ledges may also alternate at different position around the plunger shaft 122. For example, the radial ledges may be staggered at 90 degree intervals around the plunger shaft. Thus every other radial ledge—or every other set of radial ledges located at a particular location along the length of the plunger shaft—may alternate be located at angular degrees of zero, 90, zero, 90, etc degrees along the plunger shaft. At least one anti-reverse feature 128 may be provided on plunger shaft 122 to prevent pulling the plunger out of the cylinder 110. For example, anti-reverse feature 128 may be a metal tang or barb that will grip or dig into the inner surface of the cylinder to prevent pulling the plunger shaft 122 from the cylinder 110. Other types of anti-reverse feature may be used, including ratchet features interacting between plunger shaft 122 and cylinder 110. An anti-reverse feature may be provided in the stopper or attached to the stopper. An example of an anti-reverse device is described in U.S. Pat. No. 8,287,491 to Burns et al.

It should be understood that an anti-reverse feature is optional. However, use of such a feature would prevent drawing air back into the cylinder, which could contaminate the fluid, or could result in subsequent delivery of only a partial dose is some air is expelled along with fluid. On the other hand, even if air is drawn into the cylinder, consistent doses may still likely be expelled if the dispensing device is held upright, with the outlet valve 150 at the lower end of the dispensing device.

A stopper 130 may be mounted on the distal end of plunger 120. The stopper 130 may be made from rubber, plastic, or elastomeric material and may be sized and shaped to provide a close, fluid-tight fit against the inside of cylinder 110.

A collar 140 may be provided to fit around the plunger shaft 122 between the cylinder 110 and the thumb-engaging feature 124. The collar may interact with the radial ledges as described below to allow precise doses of fluid to be expelled from dispensing device 100. Collar 140 may have an inside diameter 142 that may closely fit to the outside diameter of plunger shaft 122. However, portions of the inside diameter of collar 140 may be provided as passageways 144 with a diameter large enough to enable radial ledges 126 to pass through collar 140.

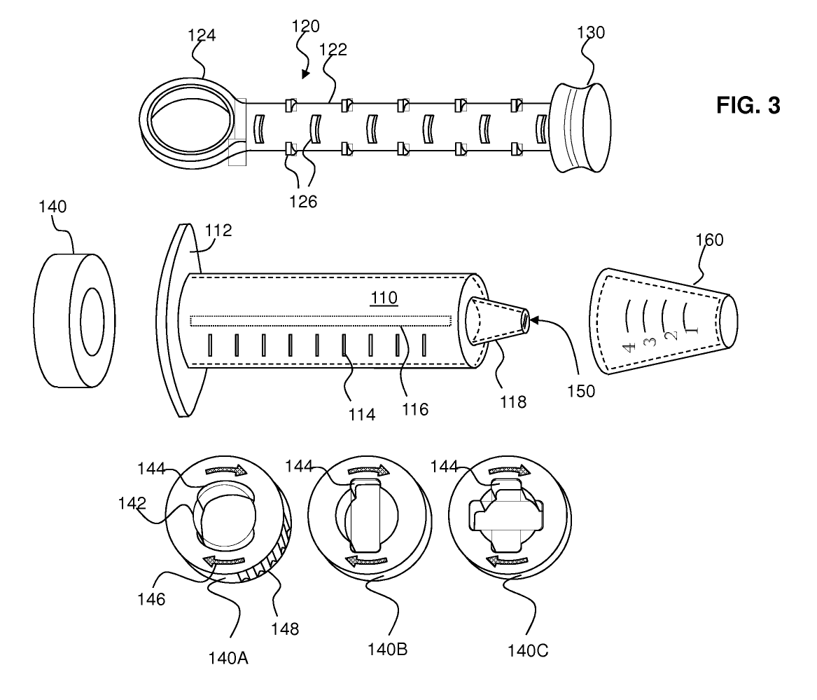

Collar 140 may have various forms, such as collars 140A, 140B, and 140C shown in FIG. 3. In the case of collar 140A, a pair of passageways 144 may be provided having a rounded aspect and may be located 180 degrees apart around the collar. In the case of collar 140B, passageways 144 may have a rectangular aspect and be located 180 degrees apart. In the case of collar 140C, four passageways 144 may be provided which are located 90 degrees apart.

Collar 140 may be provided with indicia 146, such as symbols or words, to suggest a desired motion of the collar, for example a rotary motion to align or misalign collar 140 with radial ledges 126. To help rotate collar 140, it may be provided with knurling 148 or other texture or raised or depressed areas for better gripping of the collar by the user.

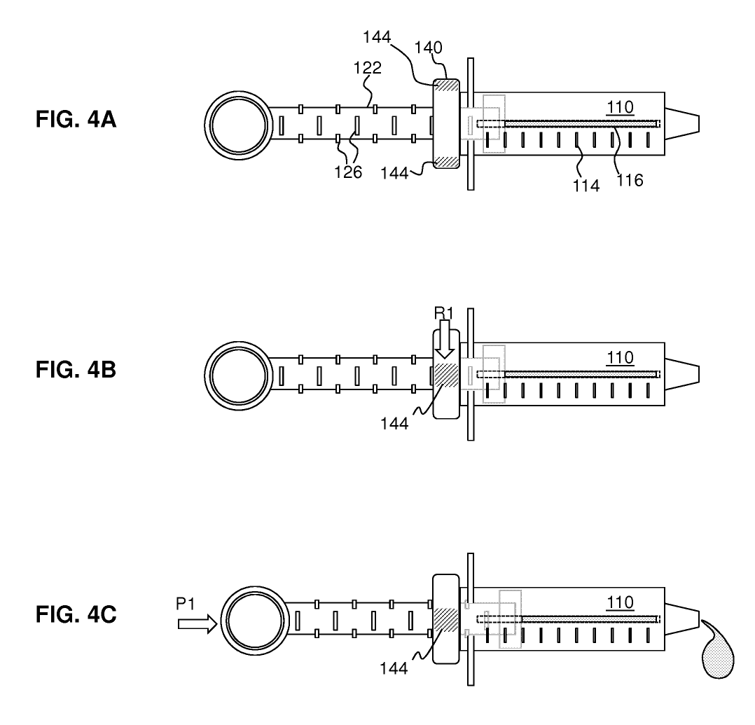

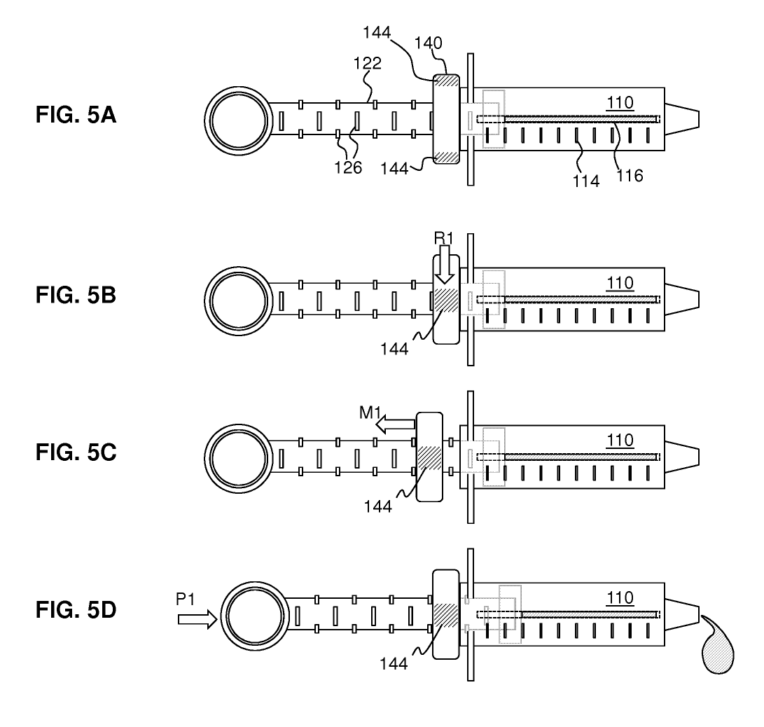

FIGS. 4A-4C illustrate use of the dispensing device. As shown in FIG. 4A, initially the dispensing device may be provided with plunger 120 mostly retracted from cylinder 110, and cylinder 110 may be substantially full of fluid as visible through transparent portion 116. Collar 140 may be positioned so that the passageways 144 are not aligned with the next radial ledge(s) 126 immediately toward the outer end of plunger 120. Thus, plunger 120 may not be pushed into cylinder 110. This may prevent inadvertent dispensing of the fluid contents.

As shown in FIG. 4B, collar 140 may be rotated as indicated by arrow R1, to bring passageways 144 into alignment with the next outward radial ledge(s) 126. Collar 140 may be attached to cylinder 110, for example by snap-on or other type of fit, preferably with the collar not removable from the cylinder. The fit of collar 140 onto cylinder 110 may include features such as detents to provide an audible or tactile indication such as a ‘click’ or a ‘bump’ at certain intervals around the circumference of the collar and/or cylinder, for example every 90 degrees around the circumference.

As shown in FIG. 4C, plunger 120 may now be pushed a short distance into cylinder 110, thereby expelling a dose of fluid from the dispensing device. In one manner of use, the user may place his thumb in thumb-engaging feature 124, and place one or more fingers below flange 112 to support the dispensing device and counter the pressure exerted by the thumb. As the plunger 120 is pressed inward, it will stop when the next outward radial ledge 126 comes into contact with the collar 140 which prevents further inward movement of plunger 120. Since the collar in this position will prevent further inward movement of the plunger, subsequent inadvertent dispensing of fluid between uses or during storage will be prevented.

The steps shown in FIGS. 4B-4C may be repeated to eventually deliver several doses, for example, about eight doses for the dispensing device shown in FIGS. 4A-4C. The user is prevented from dispensing more than one dose at a time, and the collar must be rotated between each use to permit dispensing of the next dose.

FIGS. 5A-5D illustrate use of an alternative design for the dispensing device in which the collar 140 is not fastened directly to cylinder 110. As shown in FIG. 5A, initially the dispensing device may be provided with the plunger 120 mostly retracted from cylinder 110, and cylinder 110 may be substantially full of fluid as visible through transparent portion 116. Collar 140 may be positioned so that the passageways 144 are not aligned with the next radial ledge(s) 126 immediately toward the outer end of plunger 120. Thus, plunger 120 may not be pushed into cylinder 110. This may prevent inadvertent dispensing of the fluid contents.

As shown in FIG. 5B, collar 140 may be rotated as indicated by arrow R1, to bring passageways 144 into alignment with the next outward radial ledge(s) 126.

As shown in FIG. 5C, collar 140 may be moved along plunger 120 away from cylinder 110, until the collar 140 comes up against the next outward radial ledge(s) 126.

As shown in FIG. 5D, plunger 120 may now be pushed a short distance into cylinder 110, thereby expelling a dose of fluid from the dispensing device. The plunger 120 will stop when the collar 140 once again abuts the open end of cylinder 110. Once again the collar in this position will prevent further, inadvertent dispensing of fluid.

The steps shown in FIGS. 5B-5D may be repeated to eventually deliver several doses, for example, about eight doses for the dispensing device shown in FIGS. 5A-5D. If the user wishes to deliver more than one dose at a given time, steps 5B-5C may be repeated one or more times before expelling the dose(s) as in FIG. 5D. If the collar 140 is moved further up the plunger 120 than is desired, the user may simply move the collar back down the plunger by alternately rotating the collar and moving it toward the next lower radial ledge(s).

The dispensing device prevents a user from dispensing more than one dose in one actuation of the button, which may help prevent an overdose of medication. The spacing between radial ledges on the actuator may be designed to give the precise dose, and no more, with each actuation of the device.

Although various aspects of the disclosed dispensing device have been shown and described, modifications may occur to those skilled in the art upon reading the specification.

1. A fluid dispensing device comprising:
a cylinder;
a plunger movable through said cylinder, the plunger comprising a plurality of radial ledges along the cylinder length;
a stopper attached to an inner end of said plunger, the stopper closely fitting an inside wall of the cylinder;
a collar fitted around the plunger, the collar having at least one passage for movement therethrough of a radial ledge.

2. The fluid dispensing device of claim 1, wherein the plurality of radial ledges are equally spaced along the plunger length.

3. The fluid dispensing device of claim 2, wherein the spacing of radial ledges along the plunger length corresponds to a fluid dosage to be delivered.

4. The fluid dispensing device of claim 1, wherein the plurality of radial ledges alternate between different angular positions around the plunger.

5. The fluid dispensing device of claim 4, wherein the angular positions of consecutive radial ledges along the plunger length alternate by 90 degrees around the plunger.

6. The fluid dispensing device of claim 1, further comprising an anti-reverse feature to prevent the plunger from moving outward form the cylinder.

7. The fluid dispensing device of claim 1, further comprising an outlet valve.

8. The fluid dispensing device of claim 1, wherein after the plunger is moved to dispense a dose of fluid, the collar must be rotated before the plunger can be moved to dispense a further dose of fluid.
